(19) United States
(12) Patent Application Publication
(10) Pub. No.: US 2007/0129607 A1
(43) Pub. Date: Jun. 7, 2007

(54) Laryngoscope blade

(76) Inventor: Muhammad Ashfaque, Broadstairs (GB)

Correspondence Address: LAHIVE & COCKFIELD, LLP, ONE POST OFFICE SQUARE, BOSTON, MA, 02109-2127, US

(21) Appl. No.: 11/295,823

(22) Filed: Dec. 7, 2005

(51) Int. Cl. A61B 1/267

(52) U.S. Cl. 600/194; 600/190

(57) ABSTRACT

The present invention concerns improved laryngoscope blades, particularly for use in the intubation of patients, for example orotracheal and nasotracheal intubation, and which allow viewing of a portion of the mouth, the pharynx, and the larynx of a patient, and depress the tongue and the structures of the floor of the mouth in order to prevent the tongue of the patient from obstructing the view during examination.

### BACKGROUND OF THE INVENTION

The present invention concerns improved laryngoscope blades, particularly for use in the intubation of patients, for example orotracheal and nasotracheal intubation, and which allow viewing of a portion of the mouth, the pharynx, and the larynx of a patient, and depress the tongue and the structures of the floor of the mouth in order to prevent the tongue of the patient from obstructing the view during examination.

Generally, laryngoscopes have a blade mounted on a bracket and handle for manipulation by a physician. The blade has a lower (inner, lingual) surface for engaging and compressing the tongue of a patient onto the floor of the mouth of the patient, and an upper (outer, dorsal) surface over which the physician obtains a direct line of sight through the mouth to the opening through the larynx to the trachea. A large number of laryngoscope blades are known and generally have the IPC Classification code A61B 1/267. Examples include WO 02/071930, WO 01/78583 and WO 01/66003 to name but a few. An illustration of the use of a laryngoscope and its blade is given in U.S. Pat. No. 6,494,828. An example of a laryngoscope blade having an arcuate cross-section is given by U.S. Pat. No. 6,471,643 which shows a blade having a C-section, the concave inner surface of the C-section facing upwards from (i.e. away from) the laryngoscope handle.

Laryngoscope blades are optimised to perform various tasks. For example, some are designed to reduce the risk of dental damage and to minimise the risk of harm arising to a patient as a result of any dental damage, for example of a patient aspirating a dislodged tooth. Others include the Macintosh blade (designed in 1943 by Robert Reynolds Macintosh, having a curved blade incorporating a high vertical portion with a flange facing to the left—Macintosh RR: “A new laryngoscope”, The Lancet 1:205, 1943; Macintosh RR: “Laryngoscope blades”, The Lancet: 1:485, 1944), designed for indirect lifting of the epiglottis. Blades can include a tip portion remote from the bracket on which the blade is mounted, the tip portion being for engaging the hyoid bone of the patient to effect moving of the epiglottis.

Laryngoscopes are also used to effect access to the esophagus of a patient.

An artificial airway used to intubate a patient may be a laryngeal mask airway (LMA, see for example U.S. Pat. No. 6,439,232) or an endotracheal tube (ETT). Examination afforded using a laryngoscope may be required as a result of a broken or missing tooth, the placement of a nasogastric tube, or simply the confirmation of the correct placement of an endotracheal tube. Placement of an esophageal probe (i.e. of further intubation) for cardiac output (CO) measurement, a Doppler study or Trans-esophageal Echocardiography (TEE, see for example U.S. Pat. No. 6,494,843) or for a core temperature measurement may at times be needed in an artificially ventilated patient in e.g. an operating theatre or an intensive care situation.

Also known in the art are WO 99/27840 and WO 02/11608 which disclose intubation instruments such as laryngoscopes. The intubation instruments are designed such that they have an arm which is inserted distal-end first into a patient's mouth and having an anterior surface (i.e. the lower surface referred to above) that faces a patient's tongue and floor of the mouth when inserted. The anterior surface defines a guide path for guiding the sliding movement of an endotracheal tube, the guide path including a loop protruding from the distal end of the arm, the endotracheal tube being extendable through the loop.

As can be seen from their figures, the devices are extremely large, providing no possibility of viewing the interior of the mouth etc. other than by use of the telescope incorporated therein, which is far from ideal—there is no opportunity to view areas beyond the limited field of view of the telescope. In addition, the devices hinder the further intubation of patients, which can be necessary as discussed above. Also, removal of the laryngoscope without affecting the endotracheal tube is not convenient, particularly due to the need to move the loop relative to the tube, which may frequently result in movement of the tube.

Existing laryngoscope blades are useful in effecting endotracheal intubation, but they are of limited use when a physician wishes to examine behind an already inserted endotracheal tube, for example to examine the hypopharynx. When a physician wishes to examine e.g. within the mouth, the pharynx, or the larynx of an intubated patient, the existing endotracheal tube (or laryngeal mask airway) must be manipulated in order to provide access. This is particularly the case when a physician desires to examine the throat down to the upper end of the esophagus and the posterior commissure of the larynx, or wishes to additionally intubate the patient.

### SUMMARY OF THE INVENTION

The present invention seeks to overcome the prior art disadvantages and provide improved laryngoscope blades. In particular, the laryngoscope blades of the present invention are designed to overcome the problem of effecting access to the mouth, larynx and/or pharynx of an intubated patient, and allow for the simplification of examination of an intubated patient and of further intubating a patient.

According to the present invention there is provided a laryngoscope blade comprising a body adapted for attachment to a handle downwards therefrom, and an elongate arm extending in a longitudinal direction, said elongate arm having a proximal end adjacent said body and a distal end, said elongate arm comprising a longitudinally elongate portion having a generally arcuate cross-section defining a convex upper surface, a concave lower surface, and first and second edges of said arcuate cross-section, a first flange connected to said first edge of said arcuate cross-section to define a lateral edge of said arm, a wedge connected to said second edge of said arcuate cross-section, said first flange and said wedge extending substantially laterally from said first and second edges of said arcuate cross-section, a side wall extending upwardly from said wedge, and an upper flange extending substantially laterally from said side wall.

The laryngoscope blade defines an upper face and a lower face, the convex upper surface of the longitudinally elongate portion forming part of the upper face, and the concave lower surface of the longitudinally elongate portion forming part of the lower face.

Also provided according to the present invention is a laryngoscope comprising a laryngoscope blade according to the present invention having a handle attached to and extending downwardly from said body.

Thus with the handle extending downwardly from the body, the lower face is arranged to face generally in the direction of the handle, and the upper face is arranged to face generally away from the handle.

In use the laryngoscope blades of the present invention provide a number of distinct advantages over prior art blades. Generally speaking, prior art blades are concerned with depressing the tongue of a patient in order that access can be effected to the mouth/larynx/pharynx etc. as discussed above. However, once a patient has been intubated then the mouth, larynx and pharynx can become crowded and viewing and access made difficult, as is further intubation of the patient.

Uses for the laryngoscope blades of the present invention include, but are not limited to:
(1) examination of the laryngopharynx of patients under anaesthetic and in intensive care settings;
(2) insertion of a nasogastric, nasojejunal, or an orogastric tube in a patient who already has an endotracheal tube in situ;
(3) packing the pharynx during nasal procedures behind an LMA or ETT or as otherwise necessary;
(4) packing around a non-cuffed ETT in paediatric patients;
(5) nasogastric or naso-esophageal placement of a temperature probe or a Doppler probe or TEE probe under direct vision;
(6) removal of foreign bodies from the hypopharynx;
(7) procedures involving the application of suction to secretions or blood clots from the throat under vision in an intubated patient; and
(8) endotracheal intubation.

The arcuate cross-section of the longitudinally elongate portion of the arm of the present invention is particularly useful when a physician wishes to examine the mouth, larynx and/or pharynx of an intubated patient, or when it is desired to effect further intubation of a patient. In such cases, once a patient has been intubated with a first endotracheal tube, particularly into the larynx, the laryngoscope blade of the present invention can be inserted into the patient's mouth and the first endotracheal tube located within the arcuate longitudinally elongate portion of the arm, i.e. in contact with the concave lower surface and with the patient's tongue.

Thus the present invention allows an endotracheal tube to be arranged between the tongue of a patient and the laryngoscope blade, restricting its movements and allowing free access to the mouth, larynx and pharynx, i.e. securing in place the in situ endotracheal tube

Further intubation of the patient can then be readily effected, for example it is simple and convenient to gain access to the esophagus to insert an esophageal probe and perform a Doppler study or Trans-esophageal Echocardiography.

The first flange and the wedge may vary in width in the longitudinal axis. In particular, at least one (of the first flange and the wedge) may decrease in width towards the distal end of the elongate arm.

The first flange is also referred to as comprising “wing” and “shoulder” portions, the shoulder being a proximal portion, and the wing being the rest of the flange. The function of the first flange is to act as a tongue blade when inserted into the mouth of a patient, depressing the tongue (and the floor of the mouth) as is achieved with conventional laryngoscope blades which have generally flat lower (lingual) surfaces for contacting the tongue.

At least one of the first flange and the wedge may extend substantially horizontally from the arcuate cross-section.

The first flange may be substantially co-extensive with the elongate portion, and (as shown in the Figures below) can be co-extensive with the elongate portion along substantially its whole length. As regards the wedge, it may be substantially co-extensive with the elongate portion, but it need not be co-extensive with the elongate portion for its whole length. Since it generally serves to angle the arcuate cross-section away from the body and any handle, it is required at the proximal end of the elongate arm, but is not required at the distal end of the elongate arm. Therefore it may be co-extensive with the elongate portion from its proximal end to e.g. at least half the length of the elongate portion, and can taper away from the proximal end of the arm. For example, it may have a generally triangular shape. The exact degree of co-extension required may vary between blade designs, and a suitable degree of co-extension can be readily determined by a person of ordinary skill in the art with minimum experimentation. For example, the wedge may be substantially co-extensive with the elongate portion, but may be relatively narrow towards the distal end of the arm—for example it may be less than 5, 10, 15, 20 or 25% its width at the proximal end of the arm. Thus the wedge is connected to the second edge of the arcuate cross-section along at least part of its length, and the side wall extends upwardly from the wedge or, where there is no wedge, from the second edge of the arcuate cross-section.

The arcuate cross-section of the longitudinally elongate portion with the arrangement of its concave and convex surfaces is distinct from prior art laryngoscope blades.

The longitudinally elongate portion need not be straight in the longitudinal axis, and can be curved, for example curving away from the body towards its proximal end (i.e. towards the remote end of the handle when the handle is attached). In some versions, the distal end of the elongate portion may be thickened.

Also, the arcuate cross-section of the longitudinally elongate portion may vary along its length, for example it may reduce in size towards the distal end. For example, at the proximal end the arcuate cross-section may be a 120° arc of a circle having a radius of curvature of 7.5 mm, and terminate at the distal end as a 36° arc of a circle having a radius of curvature of 17.5 mm. This is particularly useful in adult patients, and such a laryngoscope blade can be used for endotracheal intubation of a non-intubated patient, as well as being useful with intubated patients as described above. Other dimensions and arrangements, for example for different sizes of blade to suit e.g. children and young adults, will be readily apparent to one of ordinary skill in the art.

As is the case for prior art laryngoscope blades, the arm need not be flat. With a handle attached and oriented vertically downwards, the proximal end of the arm can be lower or, in a preferred embodiment, higher than the distal end. This can be effected by e.g. an arcing of the arm, or by other angulation as desired. Thus, for example, the blade can be designed either with a longitudinal curve befitting the natural curvature of the airway or as a straight instrument, in both instances bearing a furrow along its length with the concavity of the furrow facing the structures on the floor of the mouth.

The wedge also acts to contact and depress the tongue of a patient. By varying in width in the longitudinal axis, the wedge can angle the arcuate cross-section of the longitudinally elongate portion of the arm away from the body, meaning that any endotracheal tube positioned between the laryngoscope blade of the present invention and the tongue of a patient is directed away from the body and any handle attached to it.

Since the laryngoscope blades of the present invention are designed to be inserted into the mouth of a patient already containing a tube such as an endotracheal tube and to accommodate it between the blade and the tongue of the patient, the side wall (also referred to as the “web”) may be of lower height than the ones which typically form a part of prior art devices. The exact height of the side wall will depend on the general dimensions of the laryngoscope blade itself, as well as on the intended use and the dimensions of the tube already in place in the patient. For example, one for use with children will obviously need to be smaller than one for adults. Similarly, if an endotracheal tube of large diameter is already in place in the patient and has to be accommodated then it may require the side wall to be of lower height than is required for smaller diameter endotracheal tubes.

The upper laterally extending flange may extend substantially horizontally from the side wall.

The laryngoscope blade may additionally comprise light emitting means. Any suitable light emitting means may be used, for example a bulb, a light emitting diode, or a fibre optic light guide. For example a conventional bulb may be located on the side wall or between the side wall and the upper flange. Alternatively at least one LED such as a blue LED may be used to act as a light source. Similarly, a fibre optic light guide may be used, and this may be connected to light generating means remote to the laryngoscope blade. Obviously certain frequencies of light may be desired for certain applications. For example, it may be desired to stain the esophagus or larynx with a dye for diagnostic use, or with a photo-activated drug. Thus the light emitting means may emit a desired frequency of light for the dye or photo-activated drug.

Similarly, the laryngoscope blade may additionally comprise viewing means. Any means which allows an image to be gathered from the laryngoscope blade may be used, for example a CCD camera, a CMOS camera, or a fibre optic light guide. CCD and CMOS cameras are particularly useful in producing accurate high resolution images. As with the light emitting means, the viewing means may be located on the side wall, or between the side wall and the upper flange. A person of ordinary skill in the art will be readily capable of determining a suitable location for light emitting and viewing means.

As mentioned above, the field of laryngoscope blades is well developed and a large number of blades are known having mechanised sections enabling movement of the distal portion of the elongate arm relative to the proximal portion, and having sections and parts to aid in effecting movement of the epiglottis of a patient. Such modifications may be incorporated into the laryngoscope blade of the present invention.

Also provided according to the present invention is a method of restricting movement of an in situ endotracheal tube in a patient, said tube extending through said patient's mouth, comprising the step of inserting into said mouth of said patient, distal end first, a laryngoscope blade according to the present invention such that the tube is located within the arcuate longitudinally elongate portion of the arm such that it is in contact with the concave lower surface of the arcuate section and with the patient's tongue (i.e. so that it is located in the volume defined between the arcuate longitudinally elongate portion of the arm and the patient's tongue).

Also provided according to the present invention is a method of intubating a patient, comprising the step of inserting into said mouth of said patient, distal end first, a laryngoscope blade according to the present invention and intubating said patient. Such intubation may be effected via the mouth or nose. In the case of nasal intubation, the use of the laryngoscope blade of the present invention is still important in effecting viewing of the mouth and laryngopharynx, and also in moving the epiglottis of the patient. The patient may already be intubated with a first tube, and so the method may comprise locating the first tube within the arcuate longitudinally elongate portion of the arm such that it is in contact with the concave lower surface of the arcuate section and with the patient's tongue, and intubating the patient with a second tube.

### BRIEF DESCRIPTION OF THE DRAWINGS

The invention will be further apparent from the following description, with reference to the several figures of the accompanying drawings, which show, by way of example only, one form of laryngoscope blade.

Of the Figures:

FIG. 1 shows a lingual view (i.e. from below) of a laryngoscope blade of the present invention;

FIG. 2 shows a dorsal view (i.e. from above) of a laryngoscope blade of the present invention;

FIG. 3 is a schematic representation of a cross-section through lines I-I of FIG. 1; and

FIG. 4 is a schematic representation of a cross-section through lines I-I when in place in the mouth of a patient, contacting their tongue and restricting movement of a tube.

### DESCRIPTION OF ILLUSTRATED EMBODIMENT

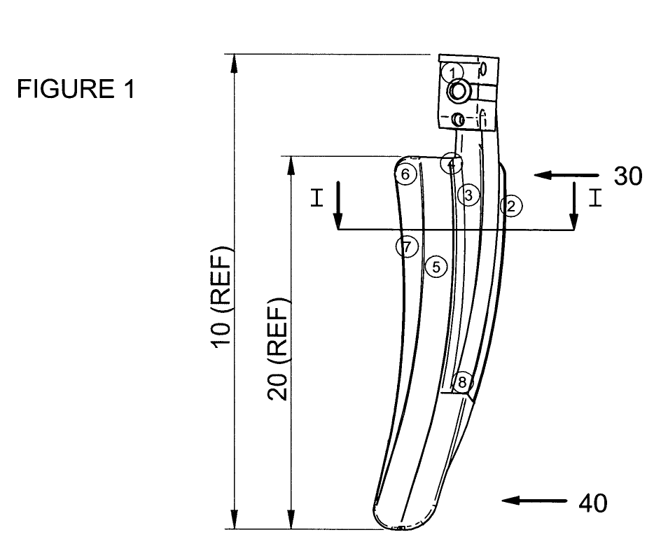

A specific example of a laryngoscope blade is shown in the accompanying Figures. As can be seen from FIG. 1, the laryngoscope blade 10 comprises a body 1 which is adapted for attachment to standard laryngoscope handles (not shown). When attached, the laryngoscope handles extend in FIG. 1 towards the viewer. Where the laryngoscope blade 10 requires a power source, for example for powering lighting means then that power may be supplied via the handle.

The blade 10 comprises an elongate arm 20 extending in a longitudinal direction, elongate arm 20 having a proximal end 30 adjacent body 1 and a distal end 40. Elongate arm 20 comprises a longitudinally elongate portion 5 (also referred to as a “furrow”) having a generally arcuate cross-section defining a convex upper surface (not shown in FIG. 1), a concave lower surface and first and second edges.

A first flange 6,7 is connected to the first edge of the arcuate cross-section 5 and defines a lateral edge of the arm. The first flange 6,7 has a first rounded portion 6 (also referred to as the “shoulder”) at its proximal end, and extends as 7 longitudinally with longitudinally elongate portion 5.

Wedge 4 is connected to the second edge of the arcuate cross-section of longitudinally elongate portion 5. This generally functions to distance the longitudinally elongate portion 5 from body 1. As such, it is not required at the distal end 40 of laryngoscope blade 10, and is co-extensive with only a portion of the longitudinally elongate portion 5, from the proximal end 30 to approximately half way between the proximal end 30 and the distal end 40.

The wedge and the first flange 4,6,7 extend laterally from the first and second edges of the arcuate cross-section of longitudinally elongate portion 5 and are generally horizontally orientated in order that they can contact the tongue of a patient and depress it. Being generally horizontal also means that force applied to the tongue is spread out, and if teeth are contacted then force applied is not localised to one specific point, reducing the chances of damaging teeth.

Side wall 3 (also referred to as the “web”) extends upwardly from wedge 4, and terminates in an upper laterally extending flange 2. This is a standard arrangement as found in other laryngoscopes.

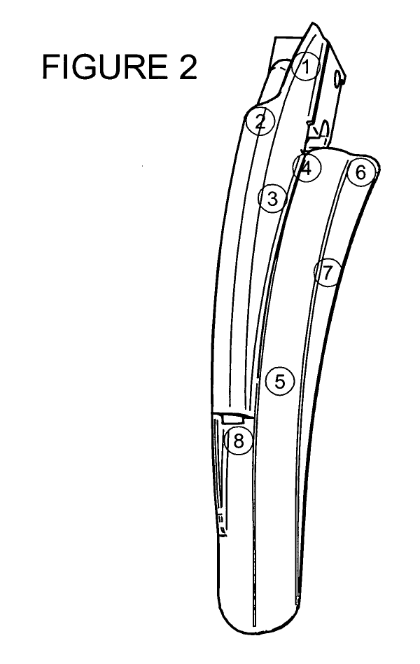

FIG. 2 shows the same features, and additionally LED lights and camera 8. The provision of lights and a camera enables the mouth and laryngopharynx to be illuminated when inserted into the mouth of a patient. Viewing may be done with the naked eye, or by way of the camera. When the camera is used then the laryngoscope may also comprise video display means to display the output of the camera, which may for example be as still photos or as moving images.

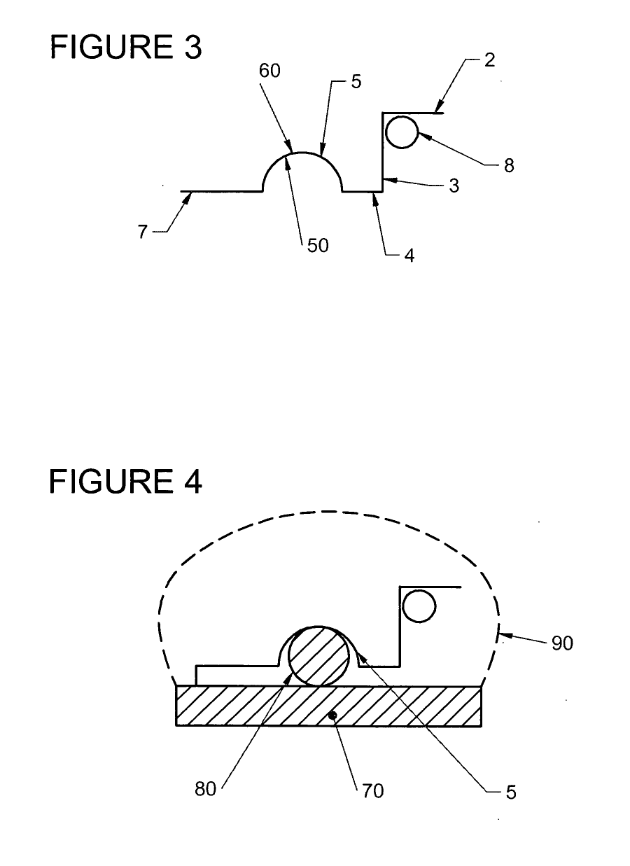

FIG. 3 shows, schematically, a cross-section through lines I-I. Lowermost is the lower (lingual) face, and uppermost is the upper (dorsal) face. As can be seen, the arcuate cross-section of longitudinally elongate portion 5 defines a convex upper surface 60 and a concave lower surface 50.

As is seen in FIG. 4, when laryngoscope blade 10 is inserted in the mouth 90 of an intubated patient, the tube 80 is restricted to the volume defined between the arcuate longitudinally elongate portion 5 of arm 20 and the patient's tongue 70. Thus clear and convenient access and viewing into the patient's mouth 90 and larynx and pharynx (not shown) is effected, the in situ ETT having been secured in place. In addition, if it is desired to further intubate the patient then despite the fact that a tube already passes through the patient's mouth 90, it does not provide an obstacle when doing the further intubation.

It will be appreciated that it is not intended to limit the invention to the above example only, many variations, such as might readily occur to one skilled in the art, being possible, without departing from the scope thereof as defined by the appended claims.

1. A laryngoscope blade, comprising a body adapted for attachment to a handle downwards therefrom, and an elongate arm extending in a longitudinal direction, said elongate arm having a proximal end adjacent said body and a distal end, said elongate arm comprising a longitudinally elongate portion having a generally arcuate cross-section defining a convex upper surface, a concave lower surface, and first and second edges of said arcuate cross-section, a first flange connected to said first edge of said arcuate cross-section to define a lateral edge of said arm, a wedge connected to said second edge of said arcuate cross-section, said first flange and said wedge extending substantially laterally from said first and second edges of said arcuate cross-section, a side wall extending upwardly from said wedge, and an upper flange extending substantially laterally from said side wall.

2. A laryngoscope blade according to claim 1, wherein the first flange is substantially co-extensive with said elongate portion.

3. A laryngoscope blade according to claim 1, wherein the wedge is substantially co-extensive with said elongate portion along at least part of the length of the elongate portion.

4. A laryngoscope blade according to claim 1, wherein said first flange or said wedge extend substantially horizontally from said arcuate cross-section.

5. A laryngoscope blade according to claim 1, wherein said upper laterally extending flange extending substantially horizontally from said side wall.

6. A laryngoscope blade according to claim 1, further comprising a light emitter.

7. A laryngoscope blade according to claim 6, wherein said light emitter is selected from the group consisting of a bulb, a light emitting diode, and a fibre optic light guide.

8. A laryngoscope blade according to claim 1, further comprising viewing means.

9. A laryngoscope blade according to claim 8, wherein said viewing means is selected from the group consisting of a CCD camera, a CMOS camera, and a fibre optic light guide.

10. A method of restricting movement of an intubated tube in a patient, said tube extending through said patient's mouth, comprising the step of inserting into said mouth of said patient, distal end first, a laryngoscope blade according to claim 1 such that said tube is located within said arcuate longitudinally elongate portion of said arm such that it is in contact with said concave lower surface of said arcuate section and with said patient's tongue.

11. A method of intubating a patient, comprising the step of inserting into said mouth of said patient, distal end first, a laryngoscope blade according to claim 1 and intubating said patient.

12. A laryngoscope, comprising a laryngoscope blade according to claim 1 having a handle attached to and extending downwardly from said body.

13. The laryngoscope according to claim 12, wherein said first flange is substantially co-extensive with said elongate portion.

14. The laryngoscope according claim 12, wherein the wedge is substantially co-extensive with said elongate portion along at least part of the length of the elongate portion.

15. The laryngoscope according to claim 12, wherein said first flange or said wedge extend substantially horizontally from said arcuate cross-section.

16. The laryngoscope according to claim 12, wherein said upper laterally extending flange extending substantially horizontally from said side wall.

17. The laryngoscope according to claim 12, further comprising a light emitter.

18. The laryngoscope according to claim 17, wherein said light emitter is selected from the group consisting of a bulb, a light emitting diode, and a fibre optic light guide.

19. The laryngoscope according to claim 12, further comprising viewing means.
